(19) United States
(12) Patent Application Publication
(10) Pub. No.: US 2026/0226525 A1
(43) Pub. Date: Aug. 6, 2026

(54) MGAT1 DEFICIENT CELLS AND USES THEREOF

(71) Applicant: Rock BioMedical, Inc., Taipei City (TW)

(72) Inventors: Chi-Huey WONG, Rancho Santa Fe, CA (US); Lan-Bo CHEN, Woburn, MA (US); Ming-Wei CHEN, Woburn, MA (US); Jeng-Shin LEE, Taipei City (TW)

(21) Appl. No.: 19/443,340

(22) Filed: Jan. 8, 2026

Related U.S. Application Data
(62) Division of application No. 18/665,736, filed on May 16, 2024, now Pat. No. 12,545,941.
(60) Provisional application No. 63/502,866, filed on May 17, 2023.

(51) Int. Cl. C12P 21/00; C12N 9/22; C12N 15/11; C12N 15/90

(52) U.S. Cl. CPC C12P 21/005; C12N 9/22; C12N 15/11; C12N 15/907; C12Y 204/01101; C12N 2310/20

(57) ABSTRACT

A method for producing a modified cell deficient in mannosyl (alpha-1,3-)-glycoprotein beta-1,2-N-acetylglucosaminyltransferase 1 (“MGAT1”) activity. Also provided is a CHO cell line deficient in MGAT1 activity produced by the method. Further disclosed are methods for producing a glycoprotein.

### CROSS-REFERENCE TO RELATED APPLICATIONS

This application claims is a divisional application of U.S. patent application Ser. No. 18/665,736, filed May 16, 2024, which benefit to and priority of U.S. Provisional Patent Applications Nos. 63/502,866, filed on May 17, 2023. The entirety of the aforementioned application is incorporated herein by reference.

### SEQUENCE LISTING

The instant application contains a Sequence Listing, which is submitted electronically in .xml format and is hereby incorporated by reference in its entirety. The .xml copy, created on May 15, 2024, is named “A1000-01200US_20240515_SeqListing.xml” and is 20,050 bytes in size.

### FIELD OF THE INVENTION

The present disclosure is directed to novel cell lines and methods of producing glycoproteins.

### BACKGROUND

Developing broadly protective vaccines against pathogenic viruses remains a major challenge in immunology and public health. For example, a broadly protective vaccine is needed to address the continued emergence of new variants of SARS-CoV2.

The SARS-CoV2 spike protein, a key immunogen, is susceptible to mutation and includes conserved epitopes shielded by glycans. Recent studies have shown that spike protein glycosylation has site-differential effects on viral infectivity, and the spike proteins generated in lung epithelial cells upon infection carry glycoforms of higher infectivity. It has also been shown that immunization of mice with spike proteins having N-linked glycans trimmed to a monoglycosylated state elicits stronger immune responses and better protection against SARS-CoV2 variants, as compared to immunization with fully glycosylated spike proteins.

One method to produce viral antigens with altered glycosylation is to mutate the O- or N-glycosylation sites of the antigens. Yet, doing so alters the protein structure and can result in loss of epitopes and thus less immunogenicity. Another method involves expressing viral antigens in cell lines engineered to have altered glycosylation enzymatic pathways.

Thus, cell lines and methods for producing glycan-modified glycoprotein antigens that can be used to develop broadly neutralizing antibodies against viruses and other pathogens are needed.

### SUMMARY

To meet the above need, a method for producing a modified cell deficient in mannosyl (alpha-1,3-)-glycoprotein beta-1,2-N-acetylglucosaminyltransferase 1 (“MGAT1”) activity is provided. The method introduces into a parent cell an RNA-guided endonuclease together with a guide RNA (“gRNA”) that comprises a sequence as set forth in GGAUGCGCAGACCUGAGCAG (SEQ ID NO: 2), GGUAGUGGAGGACGAUCUGG (SEQ ID NO: 3), UUUCUCCACCUGUAGCAGGG (SEQ ID NO: 4), or GAUCGCCAGGCACUACCGCU (SEQ ID NO: 5); culturing the parent cell; isolating a plurality of daughter cells from the cell culture; and identifying a modified cell deficient in MGAT1 activity.

Within the scope of the invention is a CHO cell line deficient in MGAT1 activity produced by the above method.

An MGAT1-deficient CHO cell is also provided in which the cell contains in its genome a nucleic acid having a sequence as set forth in SEQ ID NO: 6

A first method is disclosed for producing a glycoprotein by obtaining an MGAT1-deficient cell produced using the above method; expressing in the cell a protein having an N-glycosylation site and culturing the cell, whereby the protein is N-glycosylated at the N-glycosylation site with an oligomannose glycan; and isolating the N-glycosylated protein from the cell.

Further disclosed is a second method for producing a glycoprotein by first producing an MGAT1-deficient cell using the above method; expressing in the cell a protein having an N-glycosylation site and culturing the cell, whereby the protein is N-glycosylated at the N-glycosylation site with an oligomannose glycan; and isolating the N-glycosylated protein from the cell.

Finally, a third method for producing a glycoprotein is disclosed which is carried out by obtaining the MGAT1-deficient CHO cell mentioned above that contains SEQ ID NO: 6; expressing in the cell a protein having an N-glycosylation site and culturing the cell line, whereby the protein is N-glycosylated at the N-glycosylation site with an oligomannose glycan; and isolating the N-glycosylated protein from the cell.

The details of one or more embodiments of the invention are set forth in the description below. Other features, objects, and advantages of the invention will be apparent from the description, the drawings, and the claims.

### BRIEF DESCRIPTION OF THE DRAWINGS

FIG. 1 demonstrates a Western blot autoradiograph showing SARS-CoV2 spike proteins expressed in three different MGAT1-deficient CHO K1 cell lines and untreated (−) or treated (+) with endonuclease H (“EndoH”).

FIG. 2A demonstrates a Western blot autoradiograph examining media collected from cultures of an MGAT1-deficient CHO K1 cell line expressing SARA-CoV2 spike proteins (“S protein”) in comparison with standard proteins, including Sfg, Shm, and Smg. Sfg=fully glycosylated S protein, Shm=high mannose S protein, Smg=monoglycosylated S protein, A=medium from an adherent culture, and S=medium from a suspension culture.

FIG. 2B demonstrates a Western blot autoradiograph examining histidine-tagged S-proteins purified from a suspension culture of an MGAT1-deficient CHO K1 cell line and untreated (−) or treated (+) with EndoH in comparison with standard proteins, including Shm and Smg. Shm=high mannose S protein, Smg=monoglycosylated S protein.

FIG. 3A is a graphical presentation of the glycan composition of the 61-F.FSNVT.W fragment of the Delta His-tag-free SARS-CoV2 spike protein expressed using an exemplary cell according to the present disclosure, treated or untreated with EndoH. The untreated fragment carried glycans including HexNAc(2)Hex(4), HexNAc(2)Hex(5), HexNAc(2)Hex(6), and HexNAc(2)Hex(7), or no glycan (“un”). The treated fragment either carried no glycan or only HexNAc(1). The solid rectangle represents NAc, and the solid circle represents mannose.

FIG. 3B is a graphical presentation of the glycan composition of the 74-F.HAIHVSGTNGTK.R fragment of the Delta His-tag-free SARS-CoV2 spike protein expressed using an exemplary cell according to the present disclosure, treated or untreated with EndoH. The untreated fragment carried glycans, including HexNAc (1), HexNAc(2)Hex(3), HexNAc(2)Hex(4), and HexNAc(2)Hex(5). The treated fragment carried only HexNAc(1). The solid rectangle represents NAc, and the solid circle represents mannose.

FIG. 3C is a graphical presentation of the glycan composition of the 122-L.IVNNATNVVIK.V fragment of the Delta His-tag-free SARS-CoV2 spike protein expressed using an exemplary cell according to the present disclosure, treated or untreated with EndoH. The untreated fragment carried glycans, including HexNAc(2)Hex(4), HexNAc(2)Hex(5), HexNAc(2)Hex(6), HexNAc(2)Hex(7), and HexNAc(2)Hex(8). The treated fragment carried only HexNAc(1). The solid rectangle represents NAc, and the solid circle represents mannose.

FIG. 3D is a graphical presentation of the glycan composition of the 149-Y.YHKNNKSWMESGVY.S fragment of the Delta His-tag-free SARS-CoV2 spike protein expressed using an exemplary cell according to the present disclosure, treated or untreated with EndoH. The untreated fragment carried glycans, including HexNAc(2)Hex(5). The treated fragment carried only HexNAc(1). The solid rectangle represents NAc, and the solid circle represents mannose.

FIG. 3E is a graphical presentation of the glycan composition of the 163-Y.SSANNCTFEYVSQPF.L fragment of the Delta His-tag-free SARS-CoV2 spike protein expressed using an exemplary cell according to the present disclosure, treated or untreated with EndoH. The untreated fragment carried glycans, including HexNAc(2)Hex(5) and HexNAc(2)Hex(6). The treated fragment carried only HexNAc(1). The solid rectangle represents NAc, and the solid circle represents mannose.

FIG. 3F is a graphical presentation of the glycan composition of the 232-I.GINIT.R fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(5), HexNAc(2)Hex(6), HexNAc(2)Hex(7), HexNAc(2)Hex(8), and HexNAc(2)Hex(9) or carried no glycan (“un”). The treated fragment either carried no glycan or only HexNAc(1). The solid rectangle represents NAc, and the solid circle represents mannose.

FIG. 3G is a graphical presentation of the glycan composition of the 280-K.YNENGTIT.D fragment of the Delta His-tag-free SARS-CoV2 spike protein expressed using an exemplary cell according to the present disclosure, treated or untreated with EndoH. The untreated fragment carried glycans, including HexNAc(2)Hex(4), HexNAc(2)Hex(5), and HexNAc(2)Hex(6). The treated fragment carried only HexNAc(1). The solid rectangle represents NAc, and the solid circle represents mannose.

FIG. 3H is a graphical presentation of the glycan composition of the 329-V.RFPNIT.N fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5) or carried no glycan (“un”). The treated fragment carried no glycan or only HexNAc(1). The solid rectangle represents NAc, and the solid circle represents mannose.

FIG. 3I is a graphical presentation of the glycan composition of the 341-F.GEVFNAT.R fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5). The treated fragment carried mainly HexNAc(1) and a small portion of HexNAc(2)Hex(4). The solid rectangle represents NAc, and the solid circle represents mannose.

FIG. 3J is a graphical presentation of the glycan composition of the 601-S.VITPGTNTS.N fragment of the Delta His-tag-free SARS-CoV2 spike protein expressed using an exemplary cell according to the present disclosure, treated or untreated with EndoH. The untreated fragment carried glycans, including HexNAc(2)Hex(3), HexNAc(2)Hex(5), and HexNAc(2)Hex(6). The treated fragment carried only HexNAc(1). The solid rectangle represents NAc, and the solid circle represents mannose.

FIG. 3K is a graphical presentation of the glycan composition of the 614-V.LYQGVNCT.E fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(5), and HexNAc(2)Hex(6). The treated fragment carried mainly HexNAc(1). The solid rectangle represents NAc, and the solid circle represents mannose.

FIG. 3L is a graphical presentation of the glycan composition of the 655-V.NNSYECDIPI.G fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5) or carried no glycan (“un”). The treated fragment carried no glycan or only HexNAc(1). The solid rectangle represents NAc, and the solid circle represents mannose.

FIG. 3M is a graphical presentation of the glycan composition of the 707-A.YSNNSIA.I fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(5), HexNAc(2)Hex(6), HexNAc(2)Hex(7), and HexNAc(2)Hex(8) or carried no glycan (“un”). The treated fragment either carried no glycan or only HexNAc(1). The solid rectangle represents NAc, and the solid circle represents mannose.

FIG. 3N is a graphical presentation of the glycan composition of the 715-T.NFTIS.V fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), HexNAc(2)Hex(5), HexNAc(2)Hex(6), HexNAc(2)Hex(7), HexNAc(2)Hex(8), and HexNAc(2)Hex(9) or carried no glycan (“un”). The treated fragment either carried no glycan or only HexNAc(1). The solid rectangle represents NAc, and the solid circle represents mannose.

FIG. 3O is a graphical presentation of the glycan composition of the 799-K.TPPIKDFGGFNFS.Q fragment of the Delta His-tag-free SARS-CoV2 spike protein expressed using an exemplary cell according to the present disclosure, treated or untreated with EndoH. The untreated fragment carried glycans, including HexNAc (1), HexNAc(2)Hex(4), HexNAc(2)Hex(5), HexNAc(2)Hex(6), HexNAc(2)Hex(7), and HexNAc(2)Hex(8). The treated fragment carried only HexNAc(1). The solid rectangle represents NAc, and the solid circle represents mannose.

FIG. 3P is a graphical presentation of the glycan composition of the 1072-A.QEKNFTT.A fragment of the Delta His-tag-free SARS-CoV2 spike protein expressed using an exemplary cell according to the present disclosure, treated or untreated with EndoH. The untreated fragment carried glycans, including HexNAc (1), HexNAc(2)Hex(4), HexNAc(2)Hex(5), HexNAc(2)Hex(6), and HexNAc(2)Hex(7). The treated fragment carried only HexNAc(1). The solid rectangle represents NAc, and the solid circle represents mannose.

FIG. 3Q is a graphical presentation of the glycan composition of the 1096-S.NGTHWFVT.Q fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), HexNAc(2)Hex(5), HexNAc(2)Hex(6), and HexNAc(2)Hex(7) or carried no glycan (“un”). The treated fragment either carried no glycan or only HexNAc(1). The solid rectangle represents NAc, and the solid circle represents mannose.

FIG. 3R is a graphical presentation of the glycan composition of the 1132-V.NNTV.Y fragment of the Delta His-tag-free SARS-CoV2 spike protein expressed using an exemplary cell according to the present disclosure, treated or untreated with EndoH. The untreated fragment carried glycans, including HexNAc (1), HexNAc(2)Hex(4), HexNAc(2)Hex(5), and HexNAc(2)Hex(7). The treated fragment carried only HexNAc(1). The solid rectangle represents NAc, and the solid circle represents mannose.

FIG. 3S is a graphical presentation of the glycan composition of the 1156-K.NHTSPDVDLG.D fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5) or carried no glycan (“un”). The treated fragment either carried no glycan or only HexNAc(1). The solid rectangle represents NAc, and the solid circle represents mannose.

FIG. 3T is a graphical presentation of the glycan composition of the 1171-S.GINASVV.N fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5) or carried no glycan (“un”). The treated fragment either carried no glycan or only HexNAc(1). The solid rectangle represents NAc, and the solid circle represents mannose.

FIG. 3U is a graphical presentation of the glycan composition of the 655-V.NNSYECDIPI.G fragment of the Delta His-tag-free SARS-CoV2 spike protein expressed using an exemplary cell according to the present disclosure, treated or untreated with EndoH. The untreated fragment carried glycans, including HexNAc(1), HexNAc(2)Hex(4), and HexNAc(2)Hex(5). The treated fragment carried only HexNAc(1). The solid rectangle represents NAc, and the solid circle represents mannose.

### DETAILED DESCRIPTION

In one aspect, the present disclosure provides a method for producing a modified cell deficient in MGAT1 activity. The method comprises introducing into a parent cell, e.g., CHO K1, an RNA-guided endonuclease together with a guide RNA (gRNA).

As used herein, “MGAT1-deficient” or “deficient in MGAT1 activity” describes that the mannosyl (alpha-1,3-)-glycoprotein beta-1,2-N-acetylglucosaminyltransferase 1 of the cell does not function as compared to that of a wild-type or unmodified cell. The deficiency in MGAT-1 can be achieved by altering the MGAT-1 gene by inserting one or more nucleotides or deleting or replacing one or more nucleotides thereof, resulting in missense mutations, nonsense mutations, or frame-shift mutations of the gene. The altered gene might no longer be able to express a protein product or would express a nonfunctional protein product.

In some embodiments, the RNA-guided endonuclease is a clustered regularly interspaced short palindromic repeat-associated protein (“Cas”) that can be Streptococcus pyogenes Cas9 (“SpCas9”), enhanced SpCas9 (eSpCas9), or SpCas9-high fidelity 1 (SpCas9-HF1). In certain embodiments, the RNA-guided endonuclease is SpCas9.

Without wishing to be bound by theories, in the embodiments that the RNA-guided endonuclease is a Cas protein, the introduced gRNA will bind the target gene at a location based on the hybridization between the gRNA and the target gene. The hybridization between the gRNA and the target gene recruits the Cas protein, thereby forming a complex that activates the Cas protein. The activated Cas protein cleaves the binding site and knocks out the fragment of the target gene, leaving a double-strand break (DSB). The DSB will then be repaired via the homology-directed repair (HDR) mechanism. The repaired gene, therefore, loses a fragment thereof and is no longer intact. The gene either cannot be expressed, or the expressed gene product thereof will lose its functions.

In some embodiments, the gRNA can include a nucleic acid sequence as set forth in SEQ ID NO: 2, SEQ ID NO: 3, SEQ ID NO: 4, or SEQ ID NO: 5. In certain embodiments, the gRNA comprises a nucleic acid sequence as set forth in SEQ ID NO: 3.

In some embodiments, the parent cell of the present disclosure comprises a gene, which encodes a protein exhibiting the MGAT1 activity. In the embodiments that the gRNA comprises a nucleic acid sequence as set forth in SEQ ID NO: 2, SEQ ID NO: 3, SEQ ID NO: 4, or SEQ ID NO: 5, the gene of the parent cell, which encodes a protein exhibiting the MGAT1 activity, comprises a target sequence, which is able to hybridize with at least a portion of the SEQ ID NO: 2, SEQ ID NO: 3, SEQ ID NO: 4, and SEQ ID NO: 5 respectively.

In some embodiments, the gRNA used in the method comprises a clustered regularly interspaced short palindromic repeat RNA (“crRNA”) and a trans-activating crRNA (“tracrRNA”). The crRNA and the tracrRNA, in some embodiments, are coupled with each other via hybridization, while, in some other embodiments, the crRNA and the tracrRNA are formed into the same nucleic acid molecule. In certain embodiments, the tracrRNA comprises a nucleic acid sequence as set forth in SEQ ID NO: 11. Note that, in some embodiments, the crRNA includes a nucleic acid sequence as set forth in SEQ ID NO: 2, SEQ ID NO: 3, SEQ ID NO: 4, or SEQ ID NO: 5 and further comprises a region complementary to the 5′ end of the tracrRNA. For example, a crRNA that includes SEQ ID NO: 3 is GGUAGUGGAGGACGAUCUGGGUUUUAGAGCUAUGCU 3′ (SEQ ID NO: 13). Exemplary crRNAs that include SEQ ID NOs: 2, 4, and 5 are GGAUGCGCAGACCUGAGCAGGUUUUAGAGCUAUGCU (SEQ ID NO: 12), UUUCUCCACCUGUAGCAGGGGUUUUAGAGCUAUGCU (SEQ ID NO: 14), and GAUCGCCAGGCACUACCGCUGUUUUAGAGCUAUGCU (SEQ ID NO: 15), respectively.

In some embodiments, the RNA-guided endonuclease and the gRNA can be introduced into the parent cell, which can be a CHO cell, e.g., CHO K1, using electroporation as a pre-formed complex.

In some embodiments, the above method comprises identifying a modified cell deficient in MGAT1 activity. The identifying can be accomplished using an MGAT1 enzymatic activity assay or by sequencing a candidate cell's genomic DNA to identify those having an insertion and/or deletion that would result in loss of MGAT1 gene expression and/or loss in enzymatic activity of the MGAT1 gene product. For example, the identifying step can be carried out by performing a bacteriophage T7 Endonuclease I (“T7EI”) digestion assay on genomic DNA fragments amplified from candidate cells and sequencing the genomic DNA fragments from T7EI digestion assay-positive cells. The T7EI digestion assay is known in the art to be an effective tool for identifying small sequence mismatches. For example, DNA fragments amplified from the cell's genome DNA can be denatured and re-annealed with wild-type DNA molecules. Then, the annealed DNA fragments are digested by T7 Endonuclease I, resulting in different sizes of digested DNA fragments if mismatches exist. However, the present disclosure is not limited to using the T7EI digestion assay.

In one aspect of the present disclosure, the modified cell described above can be used to produce a glycoprotein. The first method features expressing a glycoprotein having an N-glycosylation site in an MGAT1-deficient cell (which can be a modified CHO cell according to the present disclosure). In some embodiments, the method is performed in vitro. The glycoprotein can be expressed by suitable methods known in the art, e.g., transfection, retroviral transduction, and lentivirus transduction, into the modified cell of a vector that expresses the protein. The vector can be but is not limited to, a plasmid expression vector, a retroviral vector, and a lentiviral vector. When viral vectors are employed, the viral particles can be produced in an appropriate host cell known in the art.

Expressing the glycoprotein in the MGAT1-deficient cell results in N-glycosylation at the N-glycosylation site with an oligomannose glycan. The N-glycosylated protein, i.e., an oligomannose glycoprotein, is then isolated from the cell by standard procedures known in the art. For example, the N-glycosylated protein can be purified from culture supernatant. Alternatively, the cells can be collected by centrifugation, and the N-glycosylated protein can be purified from the cell pellets.

In some embodiments, the culture of MGAT1-deficient cells can be an adherent culture or a suspension culture.

The first method for producing a glycoprotein can also comprise removing the oligomannose glycan to leave only an N-acetylglucosamine residue at the N-glycosylation site, thereby producing a monoglycosylated protein. The oligomannose glycan can be removed by incubating the glycoprotein carrying the oligomannose glycan with a glycopeptide-D-mannosyl-N4-(N-acetyl-D-glucosaminyl)2-asparagine 1,4-N-acetyl-beta-glucosaminohydrolase (“Endo H”).

In some embodiments, the glycoprotein expressed in the MGAT1-deficient cell can be, e.g., a cellular protein or a viral envelope protein.

In certain embodiments, the glycoprotein is a viral envelope protein, e.g., SARS-CoV2 spike protein, Pan-sarbecovirus spike protein, Pan-betacoronavirus spike protein, or Influenza hemagglutinin. The SARS-CoV2 spike protein can be but is not limited to alpha-SARS-CoV2, beta-SARS-CoV2, gamma-SARS-CoV2, delta-SARS-CoV2, omicron-SARS-CoV2, or variants thereof.

In certain embodiments, the SARS-CoV2 spike protein can comprise an amino acid sequence set forth in SEQ ID NO: 16 or SEQ ID NO: 17.

In other embodiments, the glycoprotein is a cellular protein such as alpha-fetoprotein, β-human chorionic gonadotropin, cancer antigens 15-3, 19-9, 27.29, 125, and 549, carcinoembryonic antigen, a carcinoembryonic antigen-related cell adhesion molecule, human epidermal growth factor receptor 2, oncofetal fibronectin, placental alkaline phosphatase, and prostate-specific antigen.

In one aspect of the present disclosure, two additional methods for producing a glycoprotein are provided. In some embodiments, these methods are performed in vitro.

The second exemplary method comprises the steps of the first method, except that the second method additionally comprises producing a modified cell deficient in MGAT1 activity.

The third exemplary method is a particular variation of the first method, in which the third method specifically employs an MGAT1-deficient cell comprising a nucleic acid in its genome, having a sequence as set forth in SEQ ID NO: 6.

The specific examples below are to be construed as merely illustrative and should not be construed as a limitation to the remainder of the disclosure in any way whatsoever. Without further elaboration, it is believed that one skilled in the art can, based on the description herein, utilize the present disclosure to its fullest extent. All publications cited herein are hereby incorporated by reference in their entirety.

### EXAMPLES

### Example 1: MGAT1 Gene Editing in CHO Cells

Exemplary adherent CHO K1 cells were cultured in Ham's F12 (Sigma) supplemented with 2 mM GLUTAMAX™ (GIBCO) and 10% fetal bovine serum (“FBS”) at 37° C., 5% CO₂. For suspension cultures, exemplary CHO K1 cells were grown in BalanCD CHO Growth A Medium (FUJIFILM Irvine Scientific) supplemented with 8 mM GLUTAMAX™ and 0.5% Anti-Clumping Supplement (FUJIFILM Irvine Scientific), also at 37° C., 5% CO₂.

### Example 2: Gene Editing

Gene editing of the MGAT1 gene in CHO K1 cells was performed using standard techniques. Briefly, ribonucleoprotein particles (“RNPs”) were prepared in vitro by mixing recombinant Cas9, universal tracrRNA (SEQ ID NO: 11), and crRNAs comprising a sequence as set forth in SEQ ID NOs: 2, 3, 4, or 5. The mixture was incubated at 37° C. for 10 min. to form the RNPs.

The RNPs (5 microliters) containing 3.3 μM Cas9, 6.6 μM tracrRNA, and 6.6 μM of one of the four crRNAs were electroporated into 1.0×10⁵ CHO K1 cells in 10 μL R buffer using the Neon system (Thermo Fisher) according to the manual. Following electroporation, cells were plated into a 24-well plate (2 mL per well) and cultured at 37° C., 5% CO₂ for three days.

### Example 3: Detection of Gene-Edited Clones by T7EI Assay

To examine the results of the gene-editing described in Example 2, electroporated cells were grown to reach a cell number of 1.0×10⁵, harvested, lysed at 37° C. for 15 min. in 100 μL lysis buffer (10 mM Tris-HCl pH 8.0, 0.05% SDS, 250 μg/ml proteinase K), and heat-inactivated at 85° C. for 15 min.

The region of interest of the MGAT1 gene was amplified from a sample of the cell lysate by polymerase chain reaction (“PCR”) using forward primer ACCCGTGAGGTGTTCCGCCT (SEQ ID NO: 7) and reverse primer AGACACGGGCAAGGAAATCCC (SEQ ID NO: 8) to give a 943 bp product.

A T7 nuclease E1 digestion assay was performed on the PCR products according to the manufacturer's protocol (New England Biolabs: “NEB”). T7E1-treated samples were analyzed by agarose gel electrophoresis to identify cells carrying an insertion/deletion (“indel”) in the MGAT1 gene region of interest.

Indel-positive cell clones were further analyzed by sequencing the 943 bp product after column purification using forward primer ACCCCCTCACCAGCCGTGAT (SEQ ID NO: 9) and reverse primer TCTGGACGAATACAGGCCCGC (SEQ ID NO: 10).

Cells were plated with serial dilutions to obtain clonal cell lines.

### Example 4: Screening for MGAT1⁻ CHO K1 Clonal Cell Lines

Loss of MGAT1 activity in CHO K1 clonal cell lines was evaluated by expressing SARS-CoV2 spike glycoprotein in the cells. The expressed spike proteins were then incubated with EndoH to detect incompletely glycosylated high-mannose-modified spike protein. Glycoproteins were expressed in CHO K1 clonal cell lines using a lentivirus expression system as outlined below.

### Lentivirus Production

1.7×10⁷ 293T cells (ATCC CRL-3216) were added to a 15 cm plate. After culturing for 24 h, the cells were transfected with four plasmid vectors totaling 30 μg as follows: 10 μg of a plasmid that expresses the viral GAG, POL, and ENV genes and carries a REV response element, 2.5 μg of a plasmid expressing REV, 2.5 μg of a plasmid expressing VSV-G protein, and 15 μg of a plasmid carrying a histidine-tagged S protein coding sequence (the wide-type S protein sequence is as set forth in SEQ ID NO: 16) and lentivirus backbone. The plasmids were transfected into the 293T cells using Lipofectamine 3000 as directed by the supplier (Thermo Fisher). Following a 48 h incubation, cell supernatants were collected, passed through a 0.45 μm filter, and concentrated 100-fold with a Lenti-X concentrator (Takara) according to the manufacturer's protocol.

### Spike Protein Production

Thirty microliters of the concentrated lentivirus were added to 2×10⁴ candidate MGAT1⁻ CHO-K1 clonal cells. After one round of single-cell limiting dilution, cells were further expanded until more than 1×10⁵ cells were reached. Then, 1×10⁵ cells were plated in a well of a 24-well plate with 0.5 mL culture medium and grown for 96 h. The supernatant from each well was collected and purified using a COVID-19 Spike Protein Affinity Resin (Repligen, SR-24156: NGL #22) according to the manual. Briefly, the collected supernatant was loaded to the column and chased for 10 mL with Buffer A (20 mM Tris-HCl, 140 mM NaCl, pH 7.5). The column was then washed for 8 CV with Buffer A and eluted for 7 CV with Buffer B (100 mM acetate, 1 M arginine, pH 5.5), followed by stripping the column for 4 CV with Buffer C (200 mM acetic acid). After that, the column was undergone a cleaning-in-place (CIP) process with Buffer D (0.1 N NaOH) for 4 CV. Then, the column was neutralized with Buffer A for 5 CV. After the purification, the collected supernatant was concentrated to a final volume of 20 μL.

### EndoH Digestion and Western Blot

Ten microliters of each concentrated supernatant containing approximately 20 μg of protein were digested with 1 μg EndoH or mock digested for 2 h at 37° C. as directed by the manufacturer (NEB). MGAT1⁻ cells produce high mannose glycoproteins that can be digested by EndoH and show fast mobility in acrylamide gel electrophoresis, i.e., a gel shift.

Following digestion, 16 μL of each sample was examined using Western blot analysis with the BOLT™ system (Thermo Fisher) with 8% Bis-Tris mini-protein gels as directed by the manufacturer.

After electrophoretic transfer from the gels to a PVDF membrane, S protein was detected using primary antibody mouse anti-SARS-CoV2 (clone 1035206; R&D Systems) and secondary antibody horseradish peroxidase-conjugated goat anti-mouse Fc (Invitrogen) then visualized using Clarity Max Western ECL substrate (BioRad).

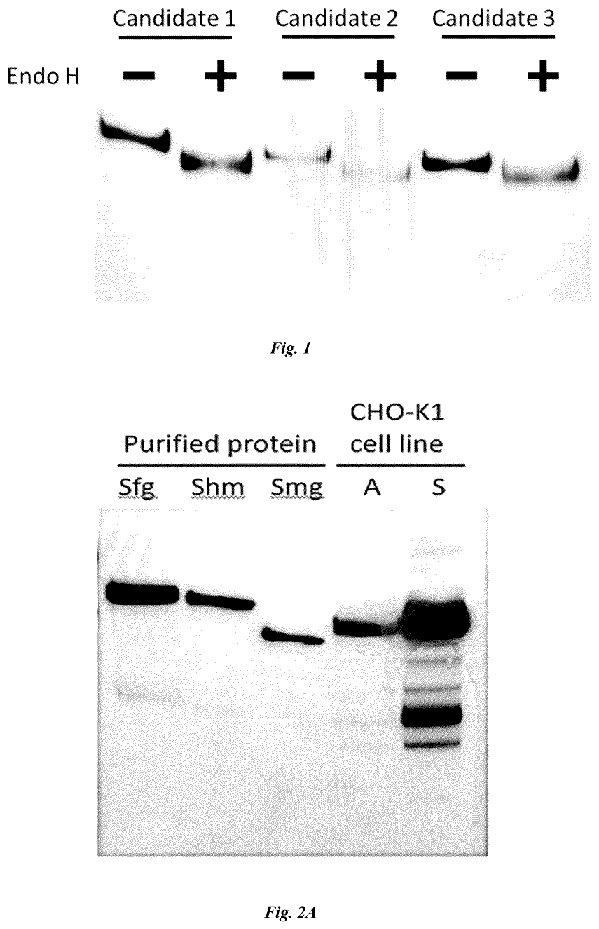

The results are shown in FIG. 1. All three candidate CHO K1 clonal cell lines showed a gel shift to lower molecular weight of the S protein after EndoH digestion, confirming the lack of MGAT1 activity in these cells.

One exemplary CHO K1 clonal cell line was selected for further assessment. The MGAT1 gene in this cell line has a two-nucleotide deletion at positions 640 and 641 of SEQ ID NO: 1 within the MGAT1 coding sequence. The two-base deletion causes a frameshift in the MGAT1 coding sequence. The DNA sequence comprising the deletion site is shown in SEQ ID NO: 6.

### Example 5: Optimization of S Protein Expression

The CHO K1 clonal cell line was adapted to suspension culture using standard methods. An adherent culture of the clonal cell line was grown in a 24-well plate, and a suspension culture was grown in a T125 flask. Histidine-tagged S protein was expressed in both cell cultures using the method set forth above.

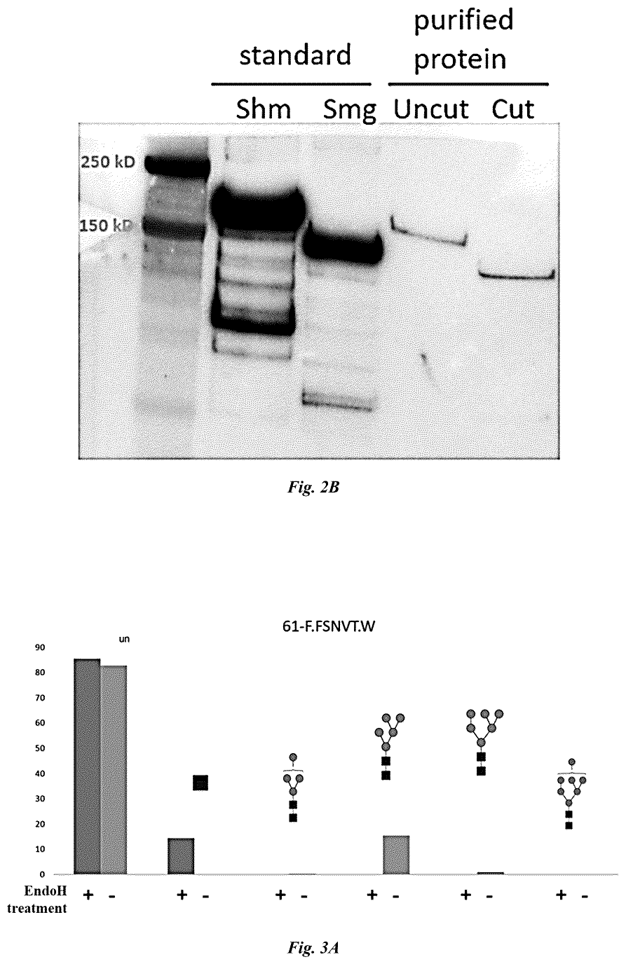

Cultured media was collected (0.5 mL from a well of the 24-well plate and 30 mL from the T125 flask), concentrated to 20 μL, and analyzed using Western blot as described above. The results are shown in FIG. 2A.

Standard S protein samples of full glycosylation (S_{fg}), high mannose glycosylation (Sₕₘ), and monoglycosylated (S_{mg}) showed different mobility as expected. See FIG. 2, first three lanes.

Both adherent (A) and suspension (S) cultures of the CHO K1 clonal cell line expressed significant amounts of S protein after lentivirus infection. See FIG. 2A, last two lanes. The mobilities of the S proteins were consistent with the high mannose form, confirming that the CHO K1 cell line is MGAT1⁻.

The histidine-tagged high-mannose S protein was purified from the suspension culture medium by nickel chelation using standard techniques, subjected to EndoH treatment as set forth in Example 4 above and analyzed using Western blot. The results are shown in FIG. 2B. The untreated S protein purified from the CHO K1 clonal cell line migrated at the same position, indicating a high-mannose S protein, while the EndoH-treated purified S protein migrated to the position corresponding to a monoglycosylated S protein.

Alternatively, the experiments described in Example 4 and Example 5 were repeated to express a His-tag-free SARS-CoV2 spike protein (Delta strain). The expressed and purified S proteins were separated into 5 parts and respectively treated with chymotrypsin, chymotrypsin and trypsin, trypsin, α-lytic protease and trypsin, and α-lytic protease. The treated samples were then vacuum-dried for MS analysis. The results show that the purity of the expressed spike protein samples was over 90%, and the protease digestion was efficient, with a digestion rate higher than 95%.

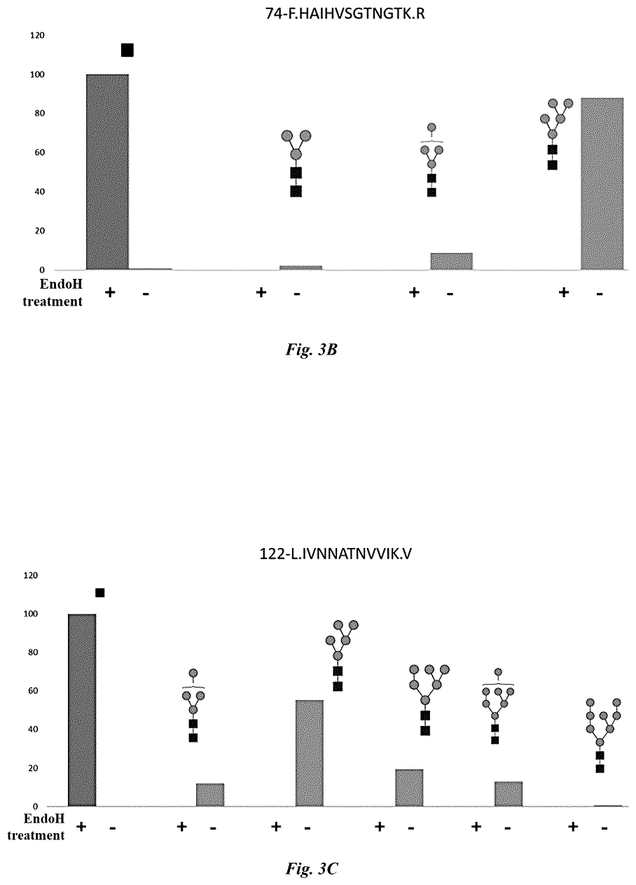

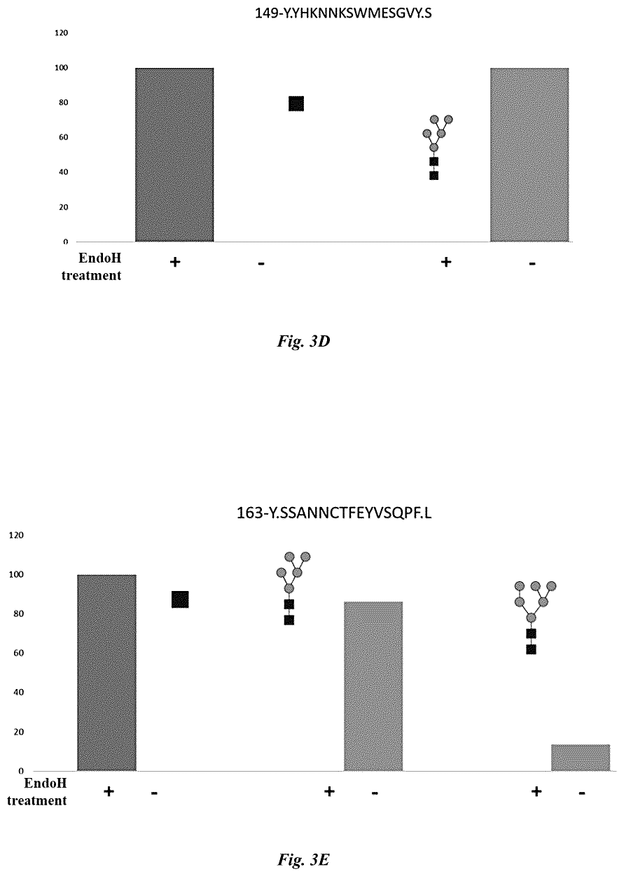

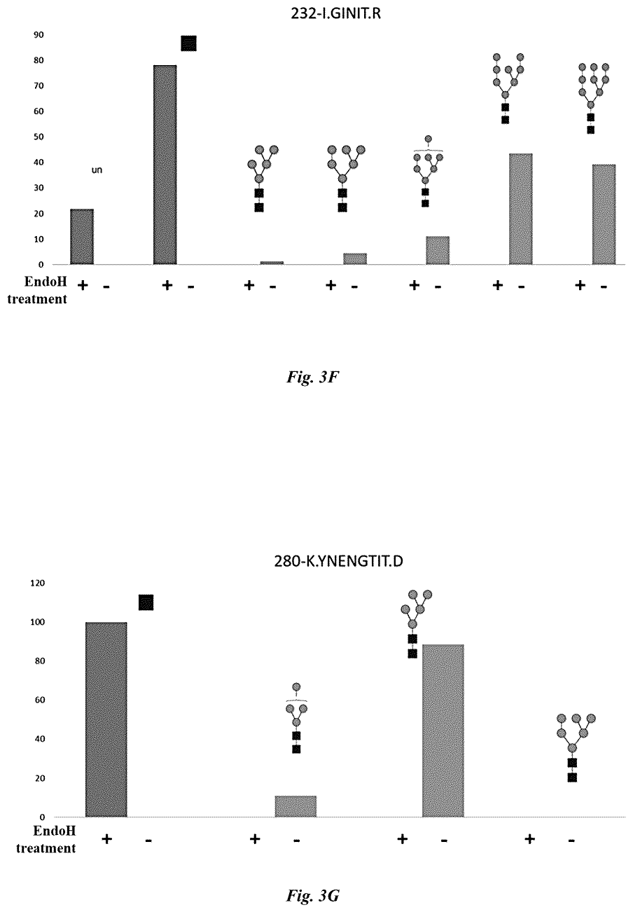

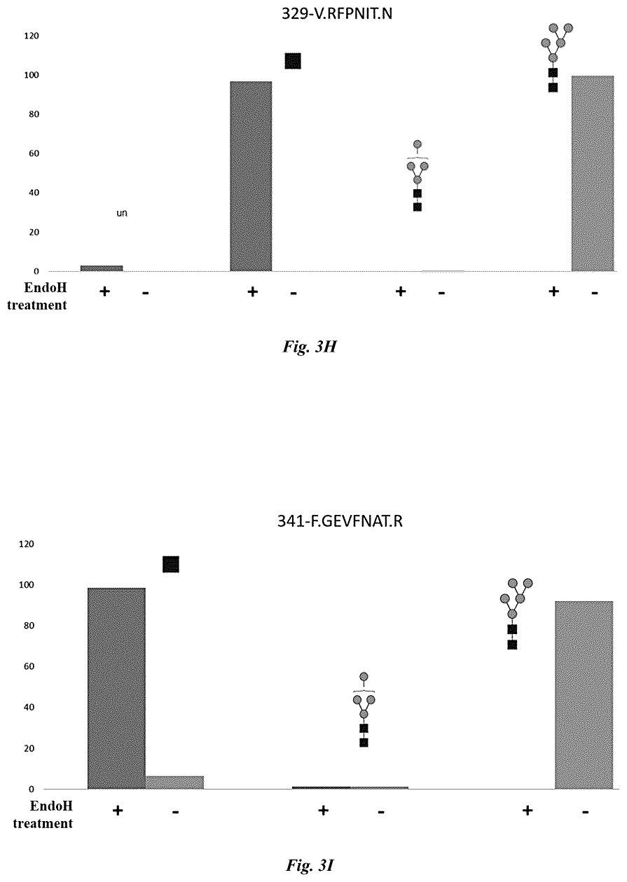

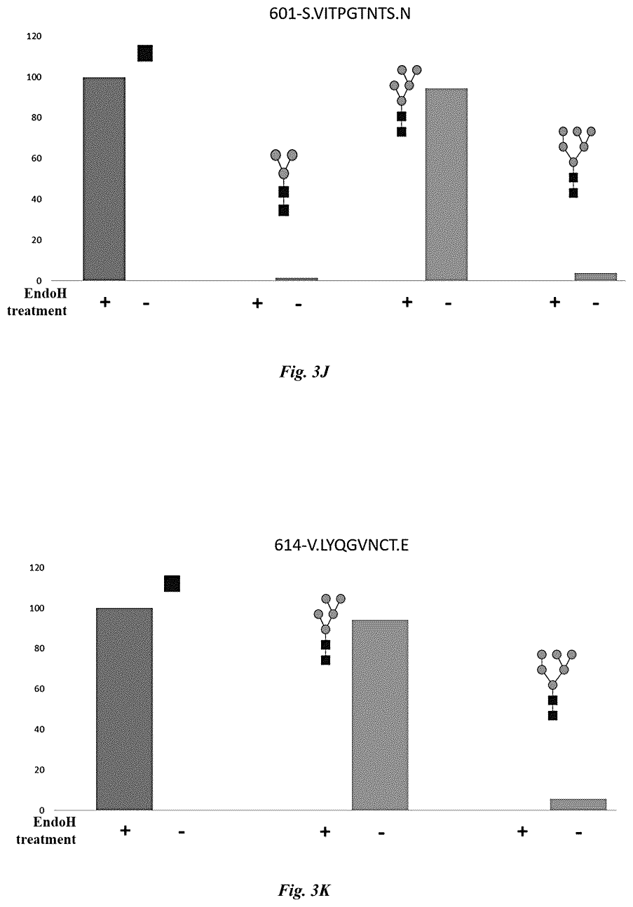

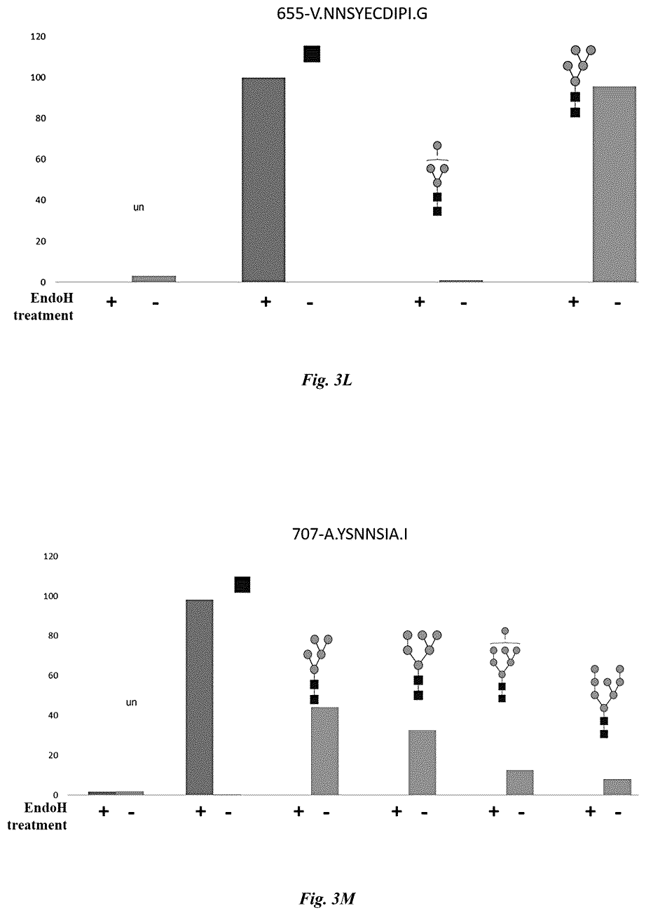

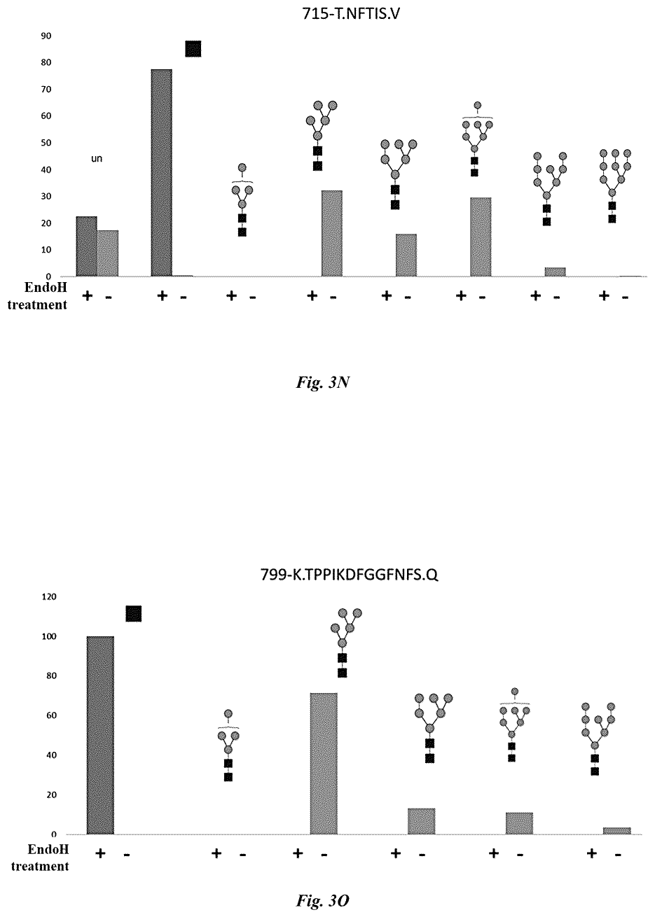

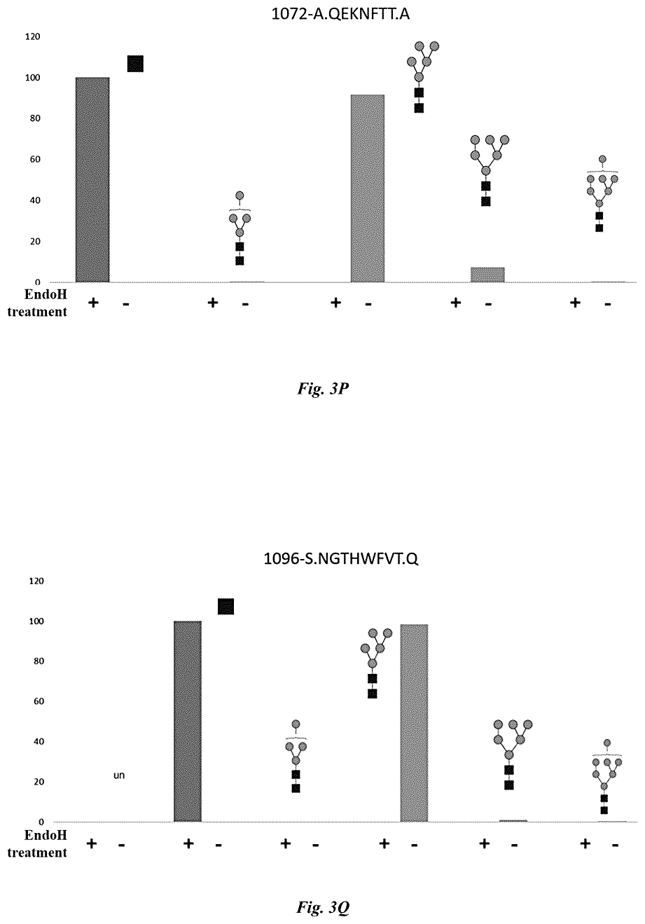

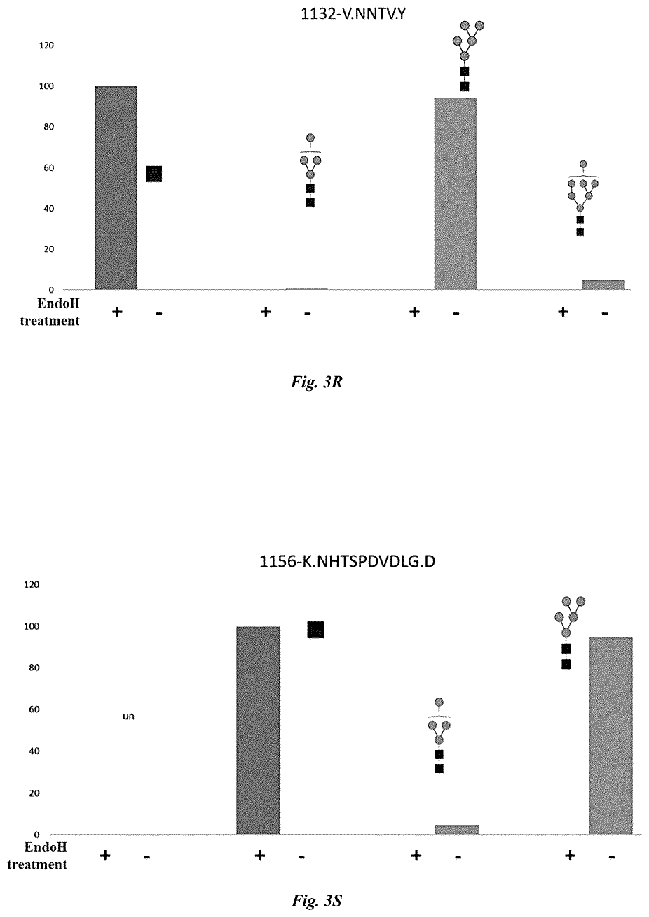

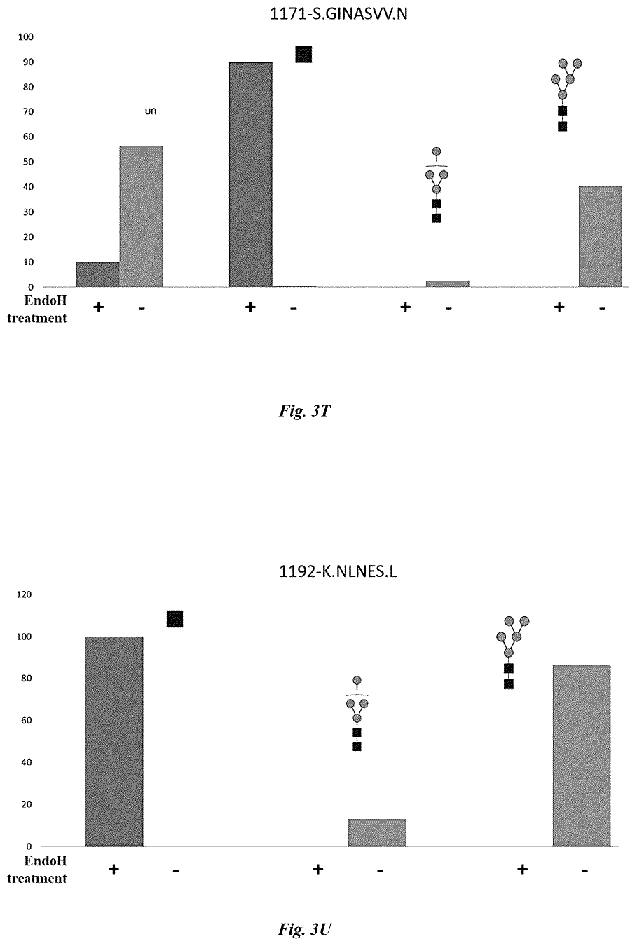

After the protease digestion, the spike proteins were cleaved into 17 peptide fragments comprising 21 N-glycosylation sites in total (the peptide comprising the N17 glycosylation site was not included). All 21 N-glycosylation sites were highly mannosylated (i.e., with high mannose contents), especially mannose-5 (Man5). The high mannose contents were further verified by using Endo H treatment as, after the treatment, only single GluNAcs were observed (FIGS. 3A to 3U).

### Other Embodiments

Embodiment 1. A method for producing a modified cell deficient in mannosyl (alpha-1,3-)-glycoprotein beta-1,2-N-acetylglucosaminyltransferase (MGAT1) activity, the method comprising: introducing into a parent cell an RNA-guided endonuclease together with a guide RNA (gRNA) comprising a sequence as set forth in GGAUGCGCAGACCUGAGCAG (SEQ ID NO: 2), GGUAGUGGAGGACGAUCUGG (SEQ ID NO: 3), UUUCUCCACCUGUAGCAGGG (SEQ ID NO: 4), or GAUCGCCAGGCACUACCGCU (SEQ ID NO: 5); culturing the parent cell and expanding the parent cell; isolating a plurality of daughter cells from the cell culture; identifying a modified cell deficient in MGAT1 activity; and isolating said modified cell deficient in MGAT1 activity.

Embodiment 2. The method of embodiment 1, wherein the identifying of a modified cell deficient in MGAT1 activity comprises: performing a bacteriophage T7 Endonuclease I (T7EI) digestion assay on genomic DNA fragments amplified from the plurality of daughter cells and sequencing the genomic DNA fragments from T7EI digestion assay-positive cells.

Embodiment 3. The method of embodiment 1 or embodiment 2, wherein the gRNA comprises a Clustered Regularly Interspaced Short Palindromic Repeat RNA (crRNA) and a trans-activating crRNA (tracrRNA), and the RNA-guided endonuclease is Cas9.

Embodiment 4. The method of any one of embodiments 1 to 3, wherein the method comprises: introducing into the cell an RNA-guided endonuclease together with a guide RNA (gRNA) using electroporation of a pre-formed complex of the RNA-guided endonuclease and the gRNA.

Embodiment 5. The method of any one of embodiments 1 to 4, wherein the parent cell is a CHO cell.

Embodiment 6. The method of embodiment 5, wherein the gRNA comprises a nucleic acid sequence as set forth in SEQ ID NO: 3.

Embodiment 7. A Chinese hamster ovary cell line produced according to the method of any one of embodiments 1 to 6.

Embodiment 8. The cell line of embodiment 7, wherein the cell line is derived from the CHO K1 cell line.

Embodiment 9. A method for producing a glycoprotein, the method comprising: obtaining a mannosyl (alpha-1,3-)-glycoprotein beta-1,2-N-acetylglucosaminyltransferase-deficient (MGAT1-deficient) cell produced according to the method of any one of embodiments 1 to 8; expressing in the MGAT1-deficient cell a protein having an N-glycosylation site, whereby the protein is N-glycosylated at the N-glycosylation site with an oligomannose glycan; and isolating the N-glycosylated protein from the cell.

Embodiment 10. The method of embodiment 9, further comprising incubating the glycosylated protein with glycopeptide-D-mannosyl-N4-(N-acetyl-D-glucosaminyl)2-asparagine 1,4-N-acetyl-beta-glucosaminohydrolase (Endo H) to remove the oligomannose glycan, whereby an N-acetylglucosamine residue remains at the N-glycosylation site.

Embodiment 11. The method of embodiment 9 or embodiment 10, wherein the protein is a viral envelope protein.

Embodiment 12. The method of embodiment 11, wherein the protein is a viral envelope protein selected from the group consisting of SARS-CoV2 spike protein, Pan-sarbecovirus spike protein, Pan-betacoronavirus spike protein, and Influenza hemagglutinin.

Embodiment 13. The method of embodiment 12, wherein the SARS-CoV2 spike protein is an alpha-SARS-CoV2 spike protein, beta-SARS-CoV2 spike protein, gamma-SARS-CoV2 spike protein, delta-SARS-CoV2 spike protein, or omicron-SARS-CoV2 spike protein.

Embodiment 14. The method of embodiment 13, wherein the SARS-CoV2 spike protein comprises an amino acid sequence as set forth in SEQ ID NO: 16 or SEQ ID NO: 17.

Embodiment 15. The method of embodiment 9 or embodiment 10, wherein the protein is selected from the group consisting of alpha-fetoprotein, β-human chorionic gonadotropin, cancer antigens 15-3, 19-9, 27.29, 125, and 549, carcinoembryonic antigen, a carcinoembryonic antigen-related cell adhesion molecule, human epidermal growth factor receptor 2, oncofetal fibronectin, placental alkaline phosphatase, and prostate-specific antigen.

Embodiment 16. A method for producing a glycoprotein, the method comprising: producing a mannosyl (alpha-1,3-)-glycoprotein beta-1,2-N-acetylglucosaminyltransferase-deficient (MGAT1-deficient) cell according to the method of any one of embodiments 1 to 15; expressing in the MGAT1-deficient cell a protein having an N-glycosylation site, whereby the protein is N-glycosylated at the N-glycosylation site with an oligomannose glycan; and isolating the N-glycosylated protein from the cell.

Embodiment 17. A Chinese hamster ovary cell line deficient in mannosyl (alpha-1,3-)-glycoprotein beta-1,2-N-acetylglucosaminyltransferase activity, the cell line comprising in its genome a nucleic acid having a sequence as set forth in SEQ ID NO: 6.

Embodiment 18. The cell line of embodiment 17, wherein the cell line is derived from the CHO K1 cell line.

Embodiment 19. A method for producing a glycoprotein, the method comprising: obtaining the mannosyl (alpha-1,3-)-glycoprotein beta-1,2-N-acetylglucosaminyltransferase-deficient Chinese Hamster Ovary cell line of embodiment 17 or embodiment 18; expressing in the cell line a protein having an N-glycosylation site, whereby the protein is N-glycosylated at the N-glycosylation site with an oligomannose glycan; and isolating the N-glycosylated protein from the cell line.

Embodiment 20. The method of embodiment 19, further comprising incubating the glycosylated protein with glycopeptide-D-mannosyl-N4-(N-acetyl-D-glucosaminyl)2-asparagine 1,4-N-acetyl-beta-glucosaminohydrolase (Endo H) to remove the oligomannose glycan, whereby an N-acetylglucosamine residue remains at the N-glycosylation site.

Embodiment 21. The method of embodiment 19 or embodiment 20, wherein the protein is a viral envelope protein.

Embodiment 22. The method of embodiment 21, wherein the protein is a viral envelope protein selected from the group consisting of SARS-CoV2 spike protein, Pan-sarbecovirus spike protein, Pan-betacoronavirus spike protein, and Influenza hemagglutinin.

Embodiment 23. The method of embodiment 22, wherein the SARS-CoV2 spike protein is an alpha-SARS-CoV2 spike protein, beta-SARS-CoV2 spike protein, gamma-SARS-CoV2 spike protein, delta-SARS-CoV2 spike protein, or omicron-SARS-CoV2 spike protein.

Embodiment 24. The method of embodiment 23, wherein the SARS-CoV2 spike protein comprises an amino acid sequence as set forth in SEQ ID NO: 16 or SEQ ID NO: 17.

Embodiment 25. The method of embodiment 19 or embodiment 20, wherein the protein is selected from the group consisting of alpha-fetoprotein, β-human chorionic gonadotropin, cancer antigens 15-3, 19-9, 27.29, 125, and 549, carcinoembryonic antigen, a carcinoembryonic antigen-related cell adhesion molecule, human epidermal growth factor receptor 2, oncofetal fibronectin, placental alkaline phosphatase, and prostate-specific antigen.

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure and, without departing from the spirit and scope thereof, can make various changes and modifications to the invention to adapt it to various usages and conditions. Thus, other embodiments are also within the claims.

SEQUENCE LISTING

| | |
|---|---|
| SEQ ID NO: 01 | atgctgaaga agcagtctgc agggcttgtg ctttggggtg ctatcctctt tgtgggctgg |
| | aatgccctgc tgctcctctt cttctggaca cgcccagccc ctggcaggcc cccctcagat |
| | agtgctatcg atgatgaccc tgccagcctc acccgtgagg tgttccgcct ggctgaggac |
| | gctgaggtgg agttggagcg gcagcggggg ctgttgcagc aaatcaggga gcatcatgct |
| | ttgtggagac agaggtggaa agtgcccacc gtggcccctc cagcctggcc ccgtgtgcct |
| | gcgaccccct caccagccgt gatccccatc ctggtcattg cctgtgaccg cagcactgtc |
| | cggcgctgct tggataagtt gttgcactat cggccctcag ctgagcattt ccccatcatt |
| | gtcagccagg actgcgggca cgaagagaca gcacaggtca ttgcttccta tggcagtgca |
| | gtcacacaca tccggcagcc agacctgagt aacatcgctg tgcccccaga ccaccgcaag |
| | ttccagggtt actacaagat cgccaggcac taccgctggg cactgggcca gatcttcaac |
| | aagttcaagt tcccagcagc tgtggtagtg gaggacgatc tggaggtggc accagacttc |
| | tttgagtact tccaggccac ctacccactg ctgagaacag acccctccct ttggtgtgtg |
| | tctgcttgga atgacaatgg caaggagcag atggtagact caagcaaacc tgagctgctc |
| | tatcgaacag acttttttcc tggccttggc tggctgctga tggctgagct gtggacagag |
| | ctggagccca agtggcccaa ggccttctgg gatgactgga tgcgcagacc tgagcagcgg |
| | aaggggcggg cctgtattcg tccagaaatt tcaagaacga tgacctttgg ccgtaagggt |
| | gtgagccatg ggcagttctt tgatcagcat cttaagttca tcaagctgaa ccagcagttc |
| | gtgtctttca cccagttgga tttgtcatac ttgcagcggg aggcttatga ccgggatttc |
| | cttgcccgtg tctatagtgc ccccctgcta caggtggaga aagtgaggac caatgatcag |
| | aaggagctgg gggaggtgcg ggtacagtac actagcagag acagcttcaa ggcctttgct |
| | aaggccctgg gtgtcatgga tgacctcaag tctggtgtcc ccagagctgg ctaccggggc |
| | gttgtcactt tccagttcag gggtcgacgt gtccacctgg cacccccaca aacctgggaa |
| | ggctatgatc ctagctggaa ttag |
| SEQ ID NO: 02 | ggaugcgcag accugagcag |
| SEQ ID NO: 03 | gguaguggag gacgaucugg |
| SEQ ID NO: 04 | uuucuccacc uguagcaggg |
| SEQ ID NO: 05 | gaucgccagg cacuaccgcu |
| SEQ ID NO: 06 | agttcaagtt cccagcagct gtggtagtgg aggacgatgg aggggcacc agacttcttt gagt |
| SEQ ID NO: 07 | acccgtgagg tgttccgcct |
| SEQ ID NO: 08 | agacacgggc aaggaaatcc c |
| SEQ ID NO: 09 | accccctcac cagccgtgat |
| SEQ ID NO: 10 | tctggacgaa tacaggcccg c |
| SEQ ID NO: 11 | agcauagcaa guuaaaauaa ggcuaguccg uuaucaacuu gaaaaagugg |
| | caccgagucg gygcuuu |
| SEQ ID NO: 12 | ggaugcgcag accugagcag guuuuagagc uaugcu |
| SEQ ID NO: 13 | gguaguggag gacgaucugg guuuuagagc uaugcu |
| SEQ ID NO: 14 | uuucuccacc uguagcaggg guuuuagagc uaugcu |
| SEQ ID NO: 15 | gaucgccagg cacuaccgcu guuuuagagc uaugcu |
| SEQ ID NO: 16 | MFVFLVLLPLVSSQCVNLTTRTQLPPAYTNSFTRGVYYPDKVFRS |
| Spike protein of | SVLHSTQDLFLPFFSNVTWFHAIHVSGTNGTKRFDNPVLPFNDGV |
| WT Strain | YFASTEKSNIIRGWIFGTTLDSKTQSLLIVNNATNVVIKVCEFQFC |
| (Wuhan strain) | NDPFLGVYYHKNNKSWMESEFRVYSSANNCTFEYVSQPFLMDL |
| | EGKQGNFKNLREFVFKNIDGYFKIYSKHTPINLVRDLPQGFSALE |
| | PLVDLPIGINITRFQTLLALHRSYLTPGDSSSGWTAGAAAYYVGY |
| | LQPRTFLLKYNENGTITDAVDCALDPLSETKCTLKSFTVEKGIYQ |
| | TSNFRVQPTESIVRFPNITNLCPFGEVFNATRFASVYAWNRKRISN |
| | CVADYSVLYNSASFSTFKCYGVSPTKLNDLCFTNVYADSFVIRG |
| | DEVRQIAPGQTGKIADYNYKLPDDFTGCVIAWNSNNLDSKVGG |
| | NYNYLYRLFRKSNLKPFERDISTEIYQAGSTPCNGVEGENCYFPL |
| | QSYGFQPTNGVGYQPYRVVVLSFELLHAPATVCGPKKSTNLVK |
| | NKCVNFNFNGLTGTGVLTESNKKFLPFQQFGRDIADTTDAVRDP |
| | QTLEILDITPCSFGGVSVITPGTNTSNQVAVLYQDVNCTEVPVAIH |
| | ADQLTPTWRVYSTGSNVFQTRAGCLIGAEHVNNSYECDIPIGAGI |
| | CASYQTQTNSPRRARSVASQSIIAYTMSLGAENSVAYSNNSIAIPT |
| | NFTISVTTEILPVSMTKTSVDCTMYICGDSTECSNLLLQYGSFCTQ |
| | LNRALTGIAVEQDKNTQEVFAQVKQIYKTPPIKDFGGFNFSQILP |
| | DPSKPSKRSFIEDLLFNKVTLADAGFIKQYGDCLGDIAARDLICA |
| | QKFNGLTVLPPLLTDEMIAQYTSALLAGTITSGWTFGAGAALQIP |
| | FAMQMAYRFNGIGVTQNVLYENQKLIANQFNSAIGKIQDSLSST |
| | ASALGKLQDVVNQNAQALNTLVKQLSSNFGAISSVLNDILSRLD |
| | KVEAEVQIDRLITGRLQSLQTYVTQQLIRAAEIRASANLAATKMS |
| | ECVLGQSKRVDFCGKGYHLMSFPQSAPHGVVFLHVTYVPAQEK |
| | NFTTAPAICHDGKAHFPREGVFVSNGTHWFVTQRNFYEPQIITTD |
| | NTFVSGNCDVVIGIVNNTVYDPLQPELDSFKEELDKYFKNHTSPD |
| | VDLGDISGINASVVNIQKEIDRLNEVAKNLNESLIDLQELGKYEQ |
| | YIKWPWYIWLGFIAGLIAIVMVTIMLCCMTSCCSCLKGCCSCGSC |
| | CKFDEDDSEPVLKGVKLHYT |
| SEQ ID NO: 17 | MFVFLVLLPL VSSQCVNLRT RTQLPPAYTN SFTRGVYYPD |
| Spike protein of | KVFRSSVLHS TQDLFLPFFS NVTWFHAIHV SGTNGTKRFD |
| Delta strain | NPVLPFNDGV YFASIEKSNI IRGWIFGTTL DSKTQSLLIV |
| | NNATNVVIKV CEFQFCNDPF LDVYYHKNNK SWMESGVYSS |
| | ANNCTFEYVS QPFLMDLEGK QGNFKNLREF VFKNIDGYFK |
| | IYSKHTPINL VRDLPQGFSA LEPLVDLPIG INITRFQTLL |
| | ALHRSYLTPG DSSSGWTAGA AAYYVGYLQP RTFLLKYNEN |
| | GTITDAVDCA LDPLSETKCT LKSFTVEKGI YQTSNFRVQP |
| | TESIVRFPNI TNLCPFGEVF NATRFASVYA WNRKRISNCV |
| | ADYSVLYNSA SFSTFKCYGV SPTKLNDLCF TNVYADSFVI |
| | RGDEVRQIAP GQTGKIADYN YKLPDDFIGC VIAWNSNNLD |
| | SKVGGNYNYR YRLFRKSNLK PFERDISTEI YQAGSKPCNG |
| | VEGFNCYFPL QSYGFQPTNG VGYQPYRVVV LSFELLHAPA |
| | TVCGPKKSTN LVKNKCVNFN FNGLTGTGVL TESNKKFLPF |
| | QQFGRDIADT TDAVRDPQTL EILDITPCSF GGVSVITPGT |
| | NTSNQVAVLY QGVNCTEVPV AIHADQLTPT WRVYSTGSNV |
| | FQTRAGCLIG AEHVNNSYEC DIPIGAGICA SYQTQTNSRG |
| | SAGSVASQSI IAYTMSLGAE NSVAYSNNSI AIPTNFTISV |
| | TTEILPVSMT KTSVDCTMYI CGDSTECSNL LLQYGSFCTQ |
| | LNRALTGIAV EQDKNTQEVF AQVKQIYKTP PIKDFGGFNF |
| | SQILPDPSKP SKRSFIEDLL FNKVTLADAG FIKQYGDCLG |
| | DIAARDLICA QKFNGLTVLP PLLTDEMIAQ YTSALLAGTI |
| | TSGWTFGAGA ALQIPFAMQM AYRENGIGVT QNVLYENQKL |
| | IANQFNSAIG KIQDSLSSTA SALGKLQNVV NQNAQALNTL |
| | VKQLSSNFGA ISSVLNDILS RLDPPEAEVQ IDRLITGRLQ |
| | SLQTYVTQQL IRAAEIRASA NLAATKMSEC VLGQSKRVDF |
| | CGKGYHLMSF PQSAPHGVVF LHVTYVPAQE KNFTTAPAIC |
| | HDGKAHFPRE GVFVSNGTHW FVTQRNFYEP QIITTDNTFV |
| | SGNCDVVIGI VNNTVYDPLQ PELDSFKEEL DKYFKNHTSP |
| | DVDLGDISGI NASVVNIQKE IDRLNEVAKN LNESLIDLQE |
| | LGKYEQYIKW PLVPRGSPGS GYIPEAPRDG QAYVRKDGEW |
| | VLLSTFLG |

What is claimed is:

1. A mannosyl (alpha-1,3-)-glycoprotein beta-1,2-N-acetylglucosaminyltransferase-deficient (MGAT1-deficient) cell line for producing a glycoprotein, comprising in its genome SEQ ID NO: 6.

2. The MGAT1-deficient cell line of claim 1, wherein the cell line is produced by:
introducing into a parent cell an RNA-guided endonuclease together with a guide RNA (gRNA) comprising GGUAGUGGAGGACGAUCUGG (SEQ ID NO: 3);
expanding the parent cell in a cell culture;
isolating a plurality of daughter cells from the cell culture;
identifying a modified cell deficient in MGAT1 activity; and
isolating said modified cell deficient in MGAT1 activity, thereby obtaining the cell line.

3. The MGAT1-deficient cell line of claim 2, wherein identifying the modified cell deficient in MGAT1 activity comprises:
performing a bacteriophage T7 Endonuclease I (T7EI) digestion assay on genomic DNA fragments amplified from the plurality of daughter cells and sequencing the genomic DNA fragments from T7EI digestion assay-positive cells.

4. The MGAT1-deficient cell line of claim 2, wherein the gRNA comprises a Clustered Regularly Interspaced Short Palindromic Repeat RNA (crRNA) and a trans-activating crRNA (tracrRNA), and the RNA-guided endonuclease is Cas9.

5. The MGAT1-deficient cell line of claim 2, wherein the RNA-guided endonuclease and the gRNA are introduced into the parent cell as a pre-formed complex using electroporation.

6. The MGAT1-deficient cell line of claim 2, wherein the parent cell is a CHO cell.

7. A mannosyl (alpha-1,3-)-glycoprotein beta-1,2-N-acetylglucosaminyltransferase-deficient (MGAT1-deficient) cell line for producing a glycoprotein, wherein the cell line comprises SEQ ID NO: 6 in its genome and a vector, and the vector expresses a glycoprotein having an N-glycosylation site.

8. The MGAT1-deficient cell line of claim 7, wherein the vector is a plasmid expression vector, a retroviral vector, or a lentiviral vector.

9. The MGAT1-deficient cell line of claim 7, wherein the glycoprotein is a viral envelope protein.

10. The MGAT1-deficient cell line of claim 9, wherein the viral envelope protein is a SARS-CoV2 spike protein, Pan-sarbecovirus spike protein, Pan-betacoronavirus spike protein, or Influenza hemagglutinin.

11. The MGAT1-deficient cell line of claim 10, wherein the SARS-CoV2 spike protein comprises SEQ ID NO: 16 or SEQ ID NO: 17.

12. The MGAT1-deficient cell line of claim 7, wherein the glycoprotein is selected from a group consisting of alpha-fetoprotein, β-human chorionic gonadotropin, cancer antigens 15-3, 19-9, 27.29, 125, and 549, carcinoembryonic antigen, carcinoembryonic antigen-related cell adhesion molecule, human epidermal growth factor receptor 2, oncofetal fibronectin, placental alkaline phosphatase, and prostate-specific antigen.

13. The MGAT1-deficient cell line of claim 7, wherein the cell line is produced by:
introducing into a parent cell an RNA-guided endonuclease together with a guide RNA (gRNA) comprising GGUAGUGGAGGACGAUCUGG (SEQ ID NO: 3);
expanding the parent cell in a cell culture;
isolating a plurality of daughter cells from the cell culture;
identifying a modified cell deficient in MGAT1 activity; and
isolating said modified cell deficient in MGAT1 activity, thereby obtaining the cell line.

14. The MGAT1-deficient cell line of claim 13, wherein identifying the modified cell deficient in MGAT1 activity comprises:
performing a bacteriophage T7 Endonuclease I (T7EI) digestion assay on genomic DNA fragments amplified from the plurality of daughter cells and sequencing the genomic DNA fragments from T7EI digestion assay-positive cells.

15. The MGAT1-deficient cell line of claim 13, wherein the gRNA comprises a Clustered Regularly Interspaced Short Palindromic Repeat RNA (crRNA) and a trans-activating crRNA (tracrRNA), and the RNA-guided endonuclease is Cas9.

16. The MGAT1-deficient cell line of claim 13, wherein the RNA-guided endonuclease and the gRNA are introduced into the parent cell as a pre-formed complex using electroporation.

17. The MGAT1-deficient cell line of claim 13, wherein the parent cell is a CHO cell.


SEQUENCE LISTING (17 sequences)

SEQ ID NO: 1 - DNA, 1344 bp, synthetic construct
  source 1..1344: mol_type = other DNA
atgctgaagaagcagtctgcagggcttgtgctttggggtgctatcctctttgtgggctggaatgccctgctgctcctcttcttctggacacgcccagcccctggcaggcccccctcagatagtgctatcgatgatgaccctgccagcctcacccgtgaggtgttccgcctggctgaggacgctgaggtggagttggagcggcagcgggggctgttgcagcaaatcagggagcatcatgctttgtggagacagaggtggaaagtgcccaccgtggcccctccagcctggccccgtgtgcctgcgaccccctcaccagccgtgatccccatcctggtcattgcctgtgaccgcagcactgtccggcgctgcttggataagttgttgcactatcggccctcagctgagcatttccccatcattgtcagccaggactgcgggcacgaagagacagcacaggtcattgcttcctatggcagtgcagtcacacacatccggcagccagacctgagtaacatcgctgtgcccccagaccaccgcaagttccagggttactacaagatcgccaggcactaccgctgggcactgggccagatcttcaacaagttcaagttcccagcagctgtggtagtggaggacgatctggaggtggcaccagacttctttgagtacttccaggccacctacccactgctgagaacagacccctccctttggtgtgtgtctgcttggaatgacaatggcaaggagcagatggtagactcaagcaaacctgagctgctctatcgaacagacttttttcctggccttggctggctgctgatggctgagctgtggacagagctggagcccaagtggcccaaggccttctgggatgactggatgcgcagacctgagcagcggaaggggcgggcctgtattcgtccagaaatttcaagaacgatgacctttggccgtaagggtgtgagccatgggcagttctttgatcagcatcttaagttcatcaagctgaaccagcagttcgtgtctttcacccagttggatttgtcatacttgcagcgggaggcttatgaccgggatttccttgcccgtgtctatagtgcccccctgctacaggtggagaaagtgaggaccaatgatcagaaggagctgggggaggtgcgggtacagtacactagcagagacagcttcaaggcctttgctaaggccctgggtgtcatggatgacctcaagtctggtgtccccagagctggctaccggggcgttgtcactttccagttcaggggtcgacgtgtccacctggcacccccacaaacctgggaaggctatgatcctagctggaattag

SEQ ID NO: 2 - RNA, 20 nt, synthetic construct
  source 1..20: mol_type = other RNA
ggatgcgcagacctgagcag

SEQ ID NO: 3 - RNA, 20 nt, synthetic construct
  source 1..20: mol_type = other RNA
ggtagtggaggacgatctgg

SEQ ID NO: 4 - RNA, 20 nt, synthetic construct
  source 1..20: mol_type = other RNA
tttctccacctgtagcaggg

SEQ ID NO: 5 - RNA, 20 nt, synthetic construct
  source 1..20: mol_type = other RNA
gatcgccaggcactaccgct

SEQ ID NO: 6 - DNA, 64 bp, synthetic construct
  source 1..64: mol_type = other DNA
agttcaagttcccagcagctgtggtagtggaggacgatggaggtggcaccagacttctttgagt

SEQ ID NO: 7 - DNA, 20 bp, synthetic construct
  source 1..20: mol_type = other DNA
acccgtgaggtgttccgcct

SEQ ID NO: 8 - DNA, 21 bp, synthetic construct
  source 1..21: mol_type = other DNA
agacacgggcaaggaaatccc

SEQ ID NO: 9 - DNA, 20 bp, synthetic construct
  source 1..20: mol_type = other DNA
accccctcaccagccgtgat

SEQ ID NO: 10 - DNA, 21 bp, synthetic construct
  source 1..21: mol_type = other DNA
tctggacgaatacaggcccgc

SEQ ID NO: 11 - RNA, 67 nt, synthetic construct
  source 1..67: mol_type = other RNA
agcatagcaagttaaaataaggctagtccgttatcaacttgaaaaagtggcaccgagtcggygcttt

SEQ ID NO: 12 - RNA, 36 nt, synthetic construct
  source 1..36: mol_type = other RNA
ggatgcgcagacctgagcaggttttagagctatgct

SEQ ID NO: 13 - RNA, 36 nt, synthetic construct
  source 1..36: mol_type = other RNA
ggtagtggaggacgatctgggttttagagctatgct

SEQ ID NO: 14 - RNA, 36 nt, synthetic construct
  source 1..36: mol_type = other RNA
tttctccacctgtagcaggggttttagagctatgct

SEQ ID NO: 15 - RNA, 36 nt, synthetic construct
  source 1..36: mol_type = other RNA
gatcgccaggcactaccgctgttttagagctatgct

SEQ ID NO: 16 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 1248 aa, synthetic construct
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASIEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRGSAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPLVPRGSPGSGYIPEAPRDGQAYVRKDGEWVLLSTFLG